(19) United States
(12) Patent Application Publication
(10) Pub. No.: US 2024/0350394 A1
(43) Pub. Date: Oct. 24, 2024

(54) A NON-THERAPEUTIC COSMETIC METHOD FOR REDUCING WRINKLES ON A SKIN SURFACE

(71) Applicant: L'Oreal, Paris (FR)

(72) Inventors: Chin Kai Lee, Kanagawa (JP); Rosario Antonelli, Chevilly Larue (FR); Shahid Naseer, Kanagawa (FR)

(73) Assignee: L'Oreal, Paris (FR)

(21) Appl. No.: 18/569,169

(22) PCT Filed: Jul. 15, 2022
(86) PCT No.: PCT/JP2022/028549

§ 371 Date: Dec. 11, 2023

(30) Foreign Application Priority Data
Jul. 16, 2021 (JP) 2021-117999
Aug. 27, 2021 (FR) 2108969

(51) Int. Cl. A61K 8/73; A61Q 19/08

(52) U.S. Cl. CPC A61K 8/735; A61Q 19/08; A61K 2800/82

(57) ABSTRACT

The present invention provides a non-therapeutic cosmetic method for treating a skin surface, said method comprising the combination of a medium with ultrasound waves, wherein the medium includes a suitable cosmetic product.

One aspect according to the present invention provides a non-therapeutic cosmetic method for reducing wrinkles on a skin surface, the method comprising the steps of applying an ultrasound transmission medium onto the skin surface to be treated, wherein the medium includes cosmetic actives/formula and applying ultrasound waves to the skin surface with the applied medium. The ultrasound waves produce acoustic output. The ultrasound waves have a frequency of 10 KHz to 50 MHz, preferably 30 KHz to 20 MHz, and more preferably 1 MHz to 10 MHz. The ultrasound waves are generated so that the ultrasound waves propagate in the skin to a maximum depth of 3 mm from the skin surface and generate a rise in temperature of the skin at the application zones.

### TECHNICAL FIELD

The present invention relates to a non-therapeutic cosmetic method for treating a skin surface, said method comprising the combination of a medium with ultrasound waves, wherein the medium includes a suitable cosmetic product. More specifically the method aims at reducing wrinkles. The present invention also relates to a kit for implementing the method and a method of using the kit.

### BACKGROUND ART

Applying cosmetic actives/formula to skin is known as one method for improving wrinkles on the skin. A non-limiting example of the cosmetic actives/formula is hyaluronic acid. Hyaluronic acid is a naturally occurring compound found in many human tissues. The human tissues includes skin, synovial fluid of joints, vitreous fluid of eye and scaffold within cartilage, etc. Among these tissues, the largest amount of hyaluronic acid in body is found in the skin.

As skin ages and is repeatedly exposed to ultraviolet rays from the sun, dermal cells of the skin decrease their production of hyaluronic acid. Additional general information about skin aging may be found in document “Hyaluronic acid, A key molecule in skin aging” published in Dermato-Endocrinology 4:3, 253-258; July-December 2012.

Hyaluronic acid is used in moisturizing and/or anti-aging products in the form of creams, lotions, tonics, masks, etc. Hyaluronic acid may include hyaluronic acid salts such as sodium hyaluronate and potassium hyaluronate. Cosmetic products including hyaluronic acid are topically applied to the skin. Therefore, a limited amount of hyaluronic acid enters into the skin, so problems where effects on wrinkles and skin texture are limited or short-term may occur.

In order to improve the effects, hyaluronic acid or a hyaluronic acid-based composition is known to be used as dermal fillers. During use, hyaluronic acid or the hyaluronic acid based composition may be injected into the skin to fill lines, folds and shallow wrinkles. However, such hyaluronic acid fillers are typically injected with a needle just below the surface of the skin, mostly in dermal, and therefore problems such as phobias of needles, risks of infections, and difficulties of sterilization may occur.

In order to overcome the disadvantages associated with the use of needles, alternative skin tightening systems have been introduced. One of the skin tightening systems is to utilize a gel-containing drug applied as a medium to deliver energy waves towards skin and thereby the energy waves may pass through the skin.

WO 2019/164836 A1 (Patent document 1) discloses using High Intensity Focused Ultrasound (HIFU) before injecting a dermal filler compound and a fat reducing compound in the dermis of skin using a needle syringe.

KR 2015/0101965 A (Patent document 2) also discloses a use of HIFU system for a skin tightening or a face lifting procedure at the dermis and deeper layers than the dermis using an ultrasound gel as a medium for transmitting the energy to the skin. The HIFU device comprises interchangeable probe heads having different penetration depths.

KR 2016/0139518 A (Patent document 3) discloses delivering HIFU at a predetermined depth from the surface of the skin using a gel medium for an aesthetic treatment at deeper layers than the dermis of skin, generating a local thermal effect during the treatment.

### SUMMARY OF INVENTION

### Technical Problem

However, HIFU therapy as disclosed in Patent documents 1 to 3 may be invasive such that a recipient feels pain. In addition, exposure to HIFU for a long period of time may induce a significant temperature increase and too strong thermal effects on the skin. Further, regarding application to facial skin, a region to be targeted for the HIFU therapy may be limited. Since the HIFU may be invasive, devices used in the HIFU therapy can only be operated by experts or dermatologists, and they are regarded as a form of medical treatment or equivalent.

Therefore, a new solution for improving wrinkles less invasively in salons and at home is required.

Accordingly, a less invasive method which can be used in salons and at home and for providing perceivable cosmetic benefits for the skin is required.

The present invention is to provide a method combining a medium including cosmetic actives/formula and ultrasound waves for facial enhancement, which are of lower energy than HIFU, a kit for implementing the method, and a method of using a kit.

### Solution to Problem

In order to solve the above-mentioned problem, one aspect of the present invention provides a non-therapeutic cosmetic method for reducing wrinkles on a skin surface, the method comprising the steps of:
applying an ultrasound transmission medium onto the skin surface to be treated, wherein the medium includes cosmetic actives/formula and
applying ultrasound waves to the skin surface with the applied medium,
wherein the ultrasound waves produce acoustic output, wherein the ultrasound waves have a frequency of 10 KHz to 50 MHz, preferably 30 KHz to 20 MHz, and more preferably 1 MHz to 10 MHz, and
wherein the ultrasound waves are generated so that the ultrasound waves propagate in the skin to a maximum depth of 3 mm from the skin surface and generate a rise in temperature of the skin at the application zones.

According to one aspect of the present invention, the ultrasound waves may be non-focused ultrasound waves. In this case, the temperature of the skin surface may be 50° C. or lower.

According to one aspect of the present invention, the ultrasound waves may be focused ultrasound waves and the ultrasound waves may generate a local rise in temperature of the skin at the focal zone in a short time. In this case, the temperature of the skin at the focal zone when applying the ultrasound waves to the skin may be more than 40° C. and 65° C. or lower, preferably more than 40° C. and 60° C. or lower, and more preferably more than 40° C. and 50° C. or lower.

According to one aspect of the present invention, the ultrasound waves may be pulsed and focused ultrasound waves. In this case, pulse energy of the ultrasound waves may be 0.001 J or more and less than 10 J, preferably 0.01 J or more and less than 5 J.

According to one aspect of the present invention, the ultrasound waves may be pulsed ultrasound waves. In this case, pulse duration of the ultrasound waves may be 150 ms or less, preferably 80 ms or less, more preferably 4 ms to 80 ms, even more preferably 4 ms to 70 ms, and yet further more preferably 4 ms to 50 ms.

According to one aspect of the present invention, the ultrasound waves may be focused and pulsed ultrasound waves. In this case, peak power of the ultrasound waves may be less than 80W, preferably 65 W or less, more preferably 15 W or less, even more preferably 5W or less, and yet further more preferably 0.1 W to 2 W.

According to one aspect of the present invention, the ultrasound waves may be continuous waves. In this case, duration time of the ultrasound waves may be 1 s or less.

According to one aspect of the present invention, the ultrasound waves may be propagated to one or more layers of the skin, which are epidermis and/or dermis.

According to one aspect of the present invention, the cosmetic actives/formula may include at least one selected from the group consisting of hyaluronic acid, polyols, vitamins, peptides, ceramides, growth factors.

According to one aspect of the present invention, the cosmetic actives/formula may include hyaluronic acid. A concentration of the hyaluronic acid may be greater than 0.1% relative to the total mass of the medium.

According to one aspect of the present invention, a molecular weight of the hyaluronic acid may be between 5 KDa and 20 MDa, preferably between 10 KDa and 2 MDa.

According to one aspect of the present invention, a dynamic viscosity of the medium may be between 4 Pas and 50 Pas.

Another aspect of the present invention provides, a kit for implementing the method according to claim 1, the kit comprising:
a device, comprising:
   an ultrasound waves unit which comprises a transducer which emits ultrasound waves:
   a housing which houses the ultrasound waves unit; and
   a head, and
a medium for perceivable cosmetic benefits of skin and for propagating the ultrasound waves, wherein the medium includes cosmetic actives/formula.

Another aspect of the present invention provides, a method of using a kit, the kit comprising:
a device, comprising:
   an ultrasound waves unit which comprises a transducer which emits ultrasound waves:
   a housing which houses the ultrasound waves unit; and
   a head,
a medium for perceivable cosmetic benefits of skin and for propagating the ultrasound waves, wherein the medium includes cosmetic actives/formula:
   wherein the method comprises steps of:
applying the medium to a surface of the skin:
generating the ultrasound waves by means of the ultrasound waves unit;
contacting the head with the skin or the medium on the skin; and
propagating the ultrasound waves through the medium to the skin;
   wherein the ultrasound waves are focused and pulsed ultrasound waves;
   wherein the ultrasound waves have a frequency of 10 KHz to 50 MHz, preferably 30 KHz to 20 MHz, and more preferably 1 MHz to 10 MHz:
   wherein the ultrasound waves have energy of 0.001 J or more and less than 10 J, preferably 0.01 J or more and less than 5 J; and
   wherein the temperature of the skin at the focal zone when applying the ultrasound waves to the skin is more than 40° C. and 65° C. or lower, preferably more than 40° C. and 60° C. or lower, and more preferably more than 40° C. and 50° C. or lower.

### Advantageous Effects of Invention

An aspect of the present invention can provide a method combining a medium including cosmetic actives/formula and ultrasound waves for facial enhancement, which are of lower energy than HIFU, a kit for implementing the method, and a method of using a kit.

### BRIEF DESCRIPTION OF DRAWINGS

FIG. 1 is a block diagram showing the method for reducing wrinkles on a skin surface of one embodiment of the invention.

FIG. 2 is a schematic view showing the configuration of a kit of one embodiment of the invention.

FIG. 3 is a block diagram showing a method of using the kit of one embodiment of the invention.

### DESCRIPTION OF EMBODIMENTS

### Method for Reducing Wrinkles on a Skin Surface

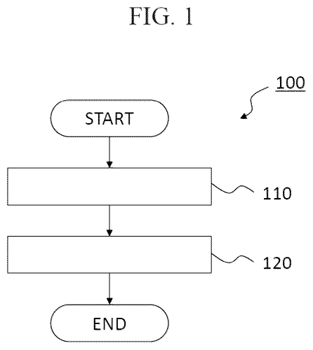

A method for reducing wrinkles on a skin surface of one embodiment of the invention will be described. The method is a non-therapeutic cosmetic method. FIG. 1 is a block diagram showing the method for reducing wrinkles on a skin surface of one embodiment of the invention. The method for reducing wrinkles on a skin surface 100 includes block 110 and block 120.

In one embodiment of the invention, at block 110, the step of applying an ultrasound transmission medium onto the skin surface to be treated is perform ed. The medium includes cosmetic actives/formula.

In one embodiment of the invention, at block 120, the step of applying ultrasound waves to the skin surface with the applied medium is performed.

In one embodiment of the invention, the ultrasound waves produce acoustic output. The ultrasound waves may be non-focused and/or focused ultrasound waves. The ultrasound waves may also be pulsed and/or continuous ultrasound waves.

In one embodiment of the invention, the ultrasound waves have a frequency of 10 KHz to 50 MHz. Preferably, the ultrasound waves have a frequency of 30 KHz to 20 MHz, and more preferably 1 MHz to 10 MHz. The ultrasound waves may have the frequency of 3 MHz to 9 MHz and may have the frequency of 5 MHz to 9 MHz. When the ultrasound waves have a frequency within such a range, the wrinkles in the skin can be less invasively and effectively improved.

In one embodiment of the invention, the ultrasound waves are generated so that the ultrasound waves propagate in the skin to a maximum depth of 3 mm from the skin surface and generate a rise in temperature of the skin at the application zones. The ultrasound waves are propagated to one or more layers of the skin, which are epidermis and/or dermis.

In another embodiment of the invention, the ultrasound waves are non-focused ultrasound waves. In this case, the temperature of the skin surface is 50° C. or lower. When the temperature of the skin surface is within such a range, the method is bearable/imperceptible in the skin for the subject who is treated using the method.

In another embodiment of the invention, the ultrasound waves are focused ultrasound waves, and the ultrasound waves generate a local rise in temperature of the skin surface at the focal zone in a short time. The term “short time” here means much shorter than 1 s, for example 40 ms. In case of the ultrasound waves being focused ultrasound waves, the temperature of the skin at the focal zone when applying the ultrasound waves to the skin is more than 40° C., and 65° C. or lower, preferably more than 40° C., and 60° C. or lower, and more preferably more than 40° C., and 50° C. or lower. When the temperature of the skin at the focal zone is within such a range, the method is bearable/imperceptible locally in the skin for the subject who is treated using the method. Given that known HIFU devices have a focal temperature of about 55° C. to 65° C., the method of one embodiment of the invention has less thermal effect on the skin and is less invasive.

In another embodiment of the invention, the ultrasound waves are pulsed and focused ultrasound waves. In this case, the ultrasound waves have pulse energy of 0.001 J or more, and less than 10 J, preferably 0.01 J or more, and less than 5 J, more preferably 0.01 J or more, and less than 1.2 J, and even more preferably 0.01 J or more, and less than 0.7 J. The focused ultrasound waves may have pulse energy of more than 0.1 J and less than 0.7 J, and may preferably more than 0.3 J and less than 0.7 J. When the ultrasound waves have the pulse energy within such a range, the load on the skin is reduced and the method of one embodiment of the invention is less invasive.

In another embodiment of the invention, the ultrasound waves are focused and pulsed ultrasound waves. In this case, the peak power of the ultrasound waves is less than 80 W, preferably 65 W or less, more preferably 15 W or less, even more preferably 5 W or less, and yet further more preferably 0.1 W to 2 W. When the peak power of the ultrasound waves is within such a range, the method can become less invasive, as well as achieve a cosmetic effect.

In further another embodiment of the invention, the ultrasound waves are pulsed ultrasound waves. In this case, the pulse duration of the ultrasound waves is 150 ms or less, preferably 80 ms or less, more preferably 4 ms to 80 ms, even more preferably 4 ms to 70 ms, and yet further more preferably 4 ms to 50 ms. When the pulse duration of the ultrasound waves is within such a range, the method can become less invasive, as well as achieve a cosmetic effect.

In addition, although embodiments using only focused ultrasound waves or pulsed ultrasound waves have been described, it goes without saying that the invention is not limited to these embodiments. Preferably, focused and pulsed ultrasound waves are used in the method. The use of focused and pulsed ultrasound waves allow a lot of energy to propagate to a limited area of the skin in a short time. In other words, the method using focused and pulsed ultrasound waves has good spatio-temporal selectivity. It should be noted that in this embodiment the temperature rises significantly up to 65° C., but the method makes it bearable/imperceptible locally in the skin.

In further another embodiment of the invention, the ultrasound waves are continuous waves. In this case, duration time of the ultrasound waves is 1 s or less.

As described above, the medium is a composition including cosmetic actives/formula. The cosmetic actives/formula may include hyaluronic acid and/or its derivatives. In exemplary embodiments, the hyaluronic acid aids in smoothing and plumping the skin. In some embodiments, the hyaluronic acid is in the form of a hydrolyzed hyaluronic acid, cross-linked hyaluronic acid, acetylated hyaluronic acid, and grafted hyaluronic acid polymer. In some embodiments, the hyaluronic acid is in the form of sodium hyaluronate or potassium hyaluronate. The hyaluronic acid may include a plurality of hyaluronic acid salts. The cosmetic actives/formula is preferably hyaluronic acid solution. The hyaluronic acid solution is preferably an aqueous hyaluronic acid solution. In one embodiment, water is present in the cosmetic actives/formula at a concentration, by weight, based on the total weight of the composition, of about 30% or greater, alternatively about 40% or greater, alternatively about 50% or greater, alternatively about 85% or less, alternatively from about 30% to about 85%, alternatively from about 40% to about 80%, alternatively from about 50% to about 75%, alternatively from about 55% to about 75%, alternatively from about 60% to about 70%, or any suitable value, range, or sub-range thereof. Thus, the water may be present in an amount from about 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, to about 85%. The medium may have the form of a gel.

In one embodiment of the invention, a viscosity of the hyaluronic acid solution depends on a molecular weight and a concentration of the hyaluronic acid. The concentration of the hyaluronic acid is preferably greater than 0.1% relative to the total mass of the medium. The concentration of hyaluronic acid is more preferably between 1.5% and 3% relative to the total mass of the medium. The employment of the concentration of the hyaluronic acid within such a range allows the wrinkles in the skin to be effectively improved.

In one embodiment of the invention, the molecular weight of the hyaluronic acid is preferably between 5 KDa and 20 MDa. The molecular weight of the hyaluronic acid is more preferably between 10 KDa and 2 Mda and further more preferably between 100 KDa and 2 MDa. The employment of the molecular weight of the hyaluronic acid within such a range increases the effect of the hyaluronic acid for the skin, and allows the epidermal wrinkles in the skin to be effectively improved.

A high concentration or high molecular weight medium may have high viscoelastic property. In one embodiment of the invention, the dynamic viscosity of the medium is preferably between 4 Pas and 50 Pas, more preferably between 20 Pas and 40 Pas. The employment of the dynamic viscosity within such a range allows the medium to not run down but to easily stay on the skin. As a result, the wrinkles in the skin can be effectively improved.

In another embodiment of the invention, the medium may include skin care actives relating to benefits such as anti-aging, whitening, hydration, and sun protection etc., which are separate from the hyaluronic acid. Non-limiting examples of the other skin care actives include polyols, vitamins, peptides, ceramides, and growth factors etc. Other examples of the skin care actives may include retinoids which include retinol and its esters, retinal, retinoic acid and its derivatives, retinoids, and in particular those described in documents FR 2570377, EP 0199636, EP0325540 and EP0402072, α-hydroxy acids such as glycolic or mandelic acid), −-hydroxy acids, α-keto acids, β-keto acids, peroxides such as benzoyl peroxide, anti-free-radical active agents such as superoxide dismutase, selenium, zinc, beta-carotenes, and tensioning polymers of natural or synthetic origin.

In exemplary embodiments, the tensioning polymers provide rapid or immediate tensing of the skin. The tensioning polymers may include, but are not limited to, pullulan, polyvinylpyrrolidone (PVP), and combinations thereof.

In yet another embodiment of the invention, the cosmetic actives/formula may include at least one selected from the group consisting of hyaluronic acid, polyols, vitamins, peptides, ceramides, growth factors, and derivatives thereof, but are not limited to this.

### (Vitamins)

Vitamins may include, but are not limited to, vitamin B3, vitamin C, vitamin E, vitamin F and derivatives thereof. As examples of vitamin B3 derivatives, mention may be made of:
esters of nicotinic acid, such as those with the following INCI names: Niacinamide Ascorbate, Niacinamide Glycolate, Niacinamide Hydroxybenzoate, Niacinamide Hydroxycitrate, Niacinamide Lactate, Niacinamide Malate, Niacinamide Mandalate, Niacinamide Salicylate, Niacinamide Thioctate, supplied by Bioderma Tech. Co LTD;
quaternary ammonium salts such as Methyl Niacinamide Chloride (INCI name) such as the commercial product MNA Chloride® from Pharmena;
the reaction products of niacinamide with polypeptides such as those derived from yeasts: INCI name Niacinamide/Yeast Polypeptide such as the commercial product sold under the trade name Vitazyme B3® by Arch Personal Care Products, L.P./Lonza Personal Care.

Use will more particularly be made of niacinamide, such as the commercial products sold under the name Niacinamide PC® (DSM Nutritional Products, Inc.) OriStar NA® (Orient Stars LLC) RonaCare Nicotinamide® (Merck KGaA/EMD Chemicals) RonaCare Nicotinamide® (EMD Chemicals)

In yet another embodiment of the invention, the cosmetic actives/formula may also include panthenol. Panthenol is an analog of pantothenic acid (vitamin B3).

### (Peptides)

In exemplary embodiments, the anti-aging peptides provide long-term biological wrinkle or line efficacy. Anti-aging peptides may include, but are not limited to, one or more acetyl hexapeptides, acetyl hexapeptide-8, one or more acetyl tetrapeptides, acetyl tetrapeptide-9, PRP (Platelet-Rich Plasma Peptides) and combinations thereof.

In other embodiments, the cosmetic actives/formula may include any one or more of acetyl hexapeptides (e.g., acetyl hexapeptide-3, -37, -38, and -51) and other acetyl tetrapep tides (e.g., acetyl tetrapeptide-2, -3, -5, -7, -8, -9, -11, and -15), and combinations thereof.

### (Ceramides)

Ceramides are a family of waxy lipid molecules that are composed of sphingosine and a fatty acid. Ceramides include or may be chosen from ceramide 1, ceramide 2, ceramide 3, ceramide 4, ceramide 5, ceramide 1A, ceramide 6II, ceramide AP, ceramide EOP, ceramide EOS, ceramide NP, ceramide NG, ceramide NS, ceramide AS, ceramide NS dilaurate, and a mixture thereof. In some instances, the ceramides include or may be chosen from ceramide-EOS, ceramide-NS, ceramide-NP, ceramide-EOH, ceramide-AS, ceramide-NH, ceramide-AP, ceramide-AH, Ceramide-OS, ceramide-OH, and a mixture thereof. In some cases, the cosmetic actives/formula may include ceramide EOP, optionally, in combination with one, two, or more other ceramides. For instance, in some instances, the cosmetic actives/formula includes a combination of ceramides, for example, a combination of ceramide EOP, ceramide NP, Ceramide AP.

### (Growth Factors)

Growth factors are typically peptides with diverse biological effects. Some growth factor families that have been identified as useful in wound healing and/or epidermal remodeling include, e.g., transforming growth factor-β (TGF-β), epidermal growth factor (EGF), insulin-like growth factors (IGFs), platelet-derived growth factor (PDGF), and fibroblast growth factors (FGFs).

Each of the skin care actives may be provided in the cosmetic actives/formula in any appropriate amount. In exemplary embodiments, the amount of each skin care actives may be between 0.001% and 30%, preferably between 0.01% and 20%, and more preferably between 0.1% and 10% based on the weight of the medium.

### (Active Ingredients)

In another embodiment of the invention, the cosmetic actives/formula may include active ingredients, which are separate from the above-mentioned skin care actives. Non-limiting examples of the one or more active agents include adenosine, 2-[4-(2-hydroxyethyl) piperazin 1-yl] ethanesulfonic acid (HEPES), hyaluronic acid, lanolin, citric acid, malic acid, lactic acid, tartaric acid, salicylic acid, vitamin C, a vitamin, a retinoid, retinal, retinoic acid, a carotenoid, an amino acid, a protein, an enzyme, and a coenzyme. In one some instances, the cosmetic actives/formula includes a humectant and moisturizing ingredients, an anti-aging gent, a depigmenting agent, an anti-wrinkle active, an agent that treats oily skin, and/or a skin penetration enhancer.

In another embodiment of the invention, the cosmetic actives/formula may include vitamin C and its derivatives and especially vitamin CG, CP and 3-0 ethyl vitamin C, alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its erivatives, ceramides and their counterparts, derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be, an extract of brown sugar (Saccharum offici narum) such as molasses extract marketed by Taiyo Kagaku under the name Liquid Molasses, without this list being exhaustive. Particular depigmenting agents include vitamin C and its derivatives and especially vitamin CG, CP and 3-0 ethyl vitamin C, alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, and an extract of Paeonia suffruticosa root, such as that sold by the company Ichimaru Pharcos under the name Botanpi Liquid B.

The term “anti-wrinkle active” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Exemplary anti-wrinkle actives may be chosen from: desquamating agents, anti-glycation agents, inhibitors of NO-synthase, agents stimulating the synthesis of dermal or epidermal macromolecules and/or preventing their degradation, agents for stimulating the proliferation of fibro blasts and/or keratinocytes, or for stimulating keratinocyte differentiation reducing agents: muscle relaxants and/or dermo-decontracting agents, and mixtures thereof.

Examples of such compounds are: adenosine and its derivatives, ascorbic acid and its derivatives such as magne sium ascorbyl phosphate and ascorbyl glucoside: tocopherol and derivatives thereof such as tocopheryl acetate, nicotinic acid and its precursors such as nicotinamide: ubiquinone: glutathione and precursors thereof such as L-2-oxothiazoli dine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soy bean proteins: algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3 methyl-} acetic acid and lipopeptides marketed by the company under the trade names SEDERMA Matrixyl 500 and Matrixyl 3000: lycopene, manganese salts and magnesium salts, especially gluconates, and mixtures thereof.

As adenosine derivatives include especially non phosphate derivatives of adenosine, such as in particular the 2′-deoxyadenosine, 2′, 3′-adenosine isopropoylidene: the toyocamycine, 1-methyladenosine, N-6-methyladenosine; adenosine N-oxide, 6-methylmercaptopurine riboside, and the 6-chloropurine riboside.

Other derivatives include adenosine receptor agonists such as adenosine adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N6-phenethyladenosine, 2-p (2-carboxy-ethyl) phenethyl-amino-5′-N-ethylcarboxamido adenosine (CGS-21680), N-ethylcarboxamido-adenosin (NECA), the 5′ (N-cyclopropyl)-carboxamidoadenosine, DPMA (PD129.944) and metrifudil.

In exemplary embodiments, the skin penetration enhancers aid in penetration of the cosmetic actives/formula into the skin. Skin penetration enhancers may include, but are not limited to, oleic acid.

Each of the active ingredients may be provided in the cosmetic actives/formula in any appropriate amount. In exemplary embodiments, the amount of each active ingredients may be between 0.01% and 0.5% based on the weight of the medium.

### (Additives)

In another embodiment of the invention, the cosmetic actives/formula may include additives, which are separate from the above-mentioned skin care actives and active ingredients. The additive may include, but is not limited to, at least one active, at least one pearl pigment, at least one polymer, at least one preservative, at least one surfactant, at least one non-water solvent, or combinations thereof.

Appropriate actives for the cosmetic actives/formula may include, but are not limited to, allantoin, bifida ferment lysate, calcium lactate, capryloyl salicylic acid, dipotassium glycyrrhizate, disodium ethylenediaminetetraacetic acid (EDTA), glycolic acid, potassium hydroxide, sodium hydroxide, trisodium ethylenediamine disuccinate, or combinations thereof.

Appropriate pearl pigments for the cosmetic actives/formula may include, but are not limited to, composite particles including a substrate formed from mica, synthetic fluorphlogopite coated with at least one mineral pigment and at least one pearl pigment, chosen from composite particles including at least one support chosen from mica, synthetic fluorphlogopite or calcium sodium borosilicate, and completely or partially coated with one or more layers of metal oxides, in particular chosen from titanium dioxide, iron oxide, tin oxide, or combinations thereof.

Appropriate polymers for the cosmetic actives/formula may include, but are not limited to, ammonium polyacryloyldimethyl taurate, carrageenan (and) potassium chloride, ceratonia siliquia (carob) gum (and) sucrose, hydroxyethyl acrylate/sodium acryloyldimethyl taurate copolymer, polyacrylamide (and) C13-C14 isoparaffin (and) Laureth-7, xanthan gum, or combinations thereof.

Appropriate preservatives for the cosmetic actives/formula may include, but are not limited to, chlorphenesin, phenoxyethanol, sodium benzoate, or combinations thereof.

Appropriate surfactants for the cosmetic actives/formula may include, but are not limited to, glyceryl stearate, myristic acid, palmitic acid, stearic acid, polyethylene glycol (PEG)-100 stearate, PEG-60 hydrogenated castor oil, seareth-100, or combinations thereof.

Appropriate non-water solvents for the cosmetic actives/formula may include, but are not limited to, denatured alcohol, caprylyl glycol, glycerin, 1,2-hexanediol, methylpropanediol, or combinations thereof.

In another further embodiment of the invention, the cosmetic actives/formula may include any number of other appropriate additives or adjuvants. Representative additives and adjuvants include, for example, water-soluble or water-miscible solvents or co-solvents, dispersion enhancing agents, colorants, fillers, antioxidants (e.g., EDTA, butylated hydroxytoluene (BHT), and tocopherol), essential oils, fragrances, dyes, neutralizing or pH-adjusting agents (e.g., citric acid, trimethylamine (TEA), and sodium hydroxide), conditioning or softening agents (e.g., panthenol and allantoin) and extracts, such as botanical extracts. Examples of anti-aging active agents or dermatological active agents include sunscreen agents (e.g., inorganic sunscreen agents, such as titanium dioxide and zinc oxide and organic sunscreen agents, such as octocrylene, ethylhexyl methoxycinnamate, and avobenzone), keratolytic agents, anti-elastase and anti-ollagenase agents, fatty acid derivatives, steroids, trace elements, extracts of planktons, enzymes and coenzymes, flavonoid, hydroxy acids and mixtures thereof, and enhancing agents.

Each of the additives may be provided in the cosmetic actives/formula in any appropriate amount. The total amount of the at least one additive may be any appropriate amount, such as, for example, about 50% or less, alternatively from about 20% to about 50%, alternatively from about 25% to about 45%, alternatively from about 25% to about 40%, alternatively from about 30% to about 35%, or any suitable value, range, or sub-range thereof, by weight, based on the weight of the medium. Thus, the additives may be present in a total amount from about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, to about 50%.

### (Others)

In one embodiment, the cosmetic actives/formula may include an exfoliation booster composition, an anti-aging booster composition, a booster additives, a base composition, an aqueous alcohol base composition, an aqueous emulsion base, and a composition.

To date, techniques have been developed to apply ultrasound waves to the skin surface. For example, WO 2019/164836 A1 discloses a technology which combines HIFU and cellulite treatment agents. WO 2019/164836 A1 achieves treatment of cellulite by injecting the agents into the dermis of the skin using a needle syringe after application of ultrasound waves. On the other hand, the method of the one embodiment of the invention simply applies the ultrasound transmission medium to the skin surface before application of ultrasound waves without using a highly invasive method such as the use of a needle syringe. Therefore, the method of the one embodiment of the present invention is less invasive than the aforementioned conventional technique. In addition, since the method is less invasive, the method can be carried out without a high degree of specialization, making it suitable for home or salon practice.

### Kit for Implementing the Method

A kit for implementing the method of one embodiment of the invention will be described. It should be noted that a kit for implementing the method of one embodiment of the invention is not limited to the following kit. Skin wrinkles are generally divided into epidermal wrinkles and dermal wrinkles. The epidermal wrinkles are characterized by being shallow and fine, and are also referred to as shallow wrinkles. The epidermal wrinkles are thought to be caused by lack of water in stratum corneum of the epidermis. On the other hand, the dermal wrinkles are thought to be caused by the loss of skin elasticity. The loss of skin elasticity is thought to be caused by a decrease or degeneration of collagen and elastin in the dermis of the skin due to damage caused by ultraviolet rays and aging, etc.

HIFU for professionals has been used to improve such wrinkles. HIFU devices focus high energy ultrasound waves on the skin. However, the HIFU devices may be invasive due to their nature of delivering the high energy ultrasound into the skin.

On the other hand, the kit used in the method of one embodiment of the invention combines ultrasound waves with a medium including cosmetic actives/formula to be used for cosmetic benefit and it may be also used to less invasively smooth the wrinkles in the skin.

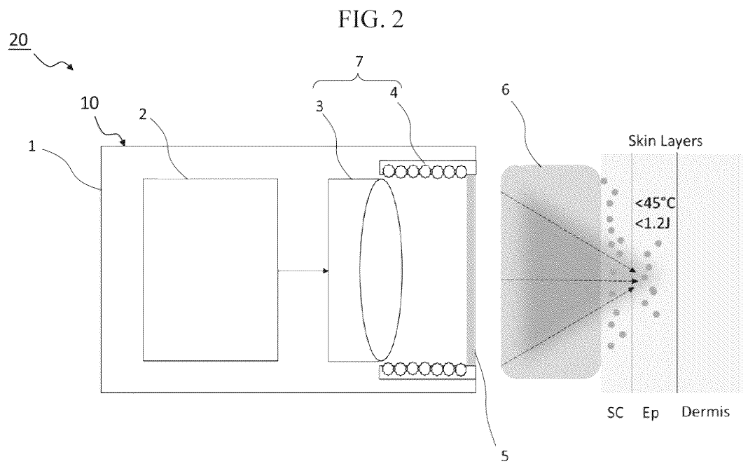

Hereinafter, a kit of one embodiment of the invention will be described with reference to the drawings. FIG. 2 is a schematic view showing the configuration of a kit of one embodiment of the invention. As shown in FIG. 2, the kit 20 comprises a cosmetic device 10 and a medium 6 including cosmetic actives/formula. In another embodiment of the invention, the device is not necessarily limited for cosmetic purpose. In another embodiment of the invention, the device is a handheld type.

As shown in FIG. 2, the cosmetic device 10 comprises an electronic unit 2, a transducer 3, a focusing unit 4, a housing 1, and a head 5. In one embodiment of the invention, an ultrasound waves unit 7 is composed of the transducer 3 and the focusing unit 4. In another embodiment of the invention, the ultrasound unit includes only a transducer. In another embodiment of the invention, the focused ultrasound waves may be produced without a focusing unit, but with the transducer alone.

In another embodiment of the invention, the cosmetic device includes a transducer, a focusing unit, a housing, and a head.

In one embodiment of the invention, the electronic unit 2 has a driving unit and a control unit (not shown).

The control unit is an interface to synchronize a selection of operation parameters such as frequency of the ultrasound waves, energy of the ultrasound waves, and/or mode of operation. In one embodiment of the invention, the control unit is used to control the driving unit based on the operation parameters.

The driving unit controlled by the control unit drives the transducer 3 and the focusing unit 4 of the ultrasound waves unit 7.

In another embodiment of the invention, the electronic unit may have a rechargeable battery unit. When the electronic unit has the rechargeable battery unit, the kit of one embodiment of the invention may be used even in a place without a power source (ex. outdoors).

In one embodiment of the invention, the transducer 3 emits ultrasound waves which are focused and pulsed ultrasound waves and have a frequency of 10 KHz to 50 MHz. As described above, the ultrasound waves are generated so that the ultrasound waves propagate in the skin to a maximum depth of 3 mm from the skin surface and generate a rise in temperature of the skin at the application zones. The transducer 3 is not particularly limited as long as it may emit ultrasound waves. The transducer 3 may be a fixed single spot-like piezoelectric transducer. When the transducer of the cosmetic device of the kit is a fixed single spot-like piezoelectric transducer, the kit can be suitably used for smaller application areas such as wrinkles at the corners of the eyes. In another embodiment of the invention, the transducer is a linearly movable single piezoelectric transducer. In another embodiment of the invention, the device may include an actuator. The actuator may move the single piezoelectric transducer linearly in a length range of 10.0 mm to 100.0 mm, and preferably 10.0 mm to 50.0 mm. The kit with the single transducer can also be suitable for the treatment of larger areas such as the cheek, front, full face and neck.

In another embodiment of the invention, the transducer is composed of an array of a plurality of fixed piezoelectric transducers. The fixed plurality of piezoelectric transducers may be arranged substantially parallel to the head of the device. When the transducer of the cosmetic device of the kit is an array of a plurality of fixed piezoelectric transducers, the kit can be suitably used for larger application areas such as the cheeks. In another embodiment of the invention, the transducer is a fixed single spot-like piezoelectric transducer, a fixed single linear piezoelectric transducer, an array of a plurality of fixed piezoelectric transducers, and/or a linearly movable single piezoelectric transducer. For example, the type of transducer may be selected depending on the shape and/or size of the areas to which the kit is applied.

In yet another embodiment of the invention, the transducer may be composed of an array of at least 20 piezoelectric transducers. In yet another embodiment of the invention, a plurality of transducers may be arranged in 1D shape (i.e., linear) or any 2D shape (i.e., matrix).

In one embodiment of the invention, the focusing unit 4 adjusts a depth of the ultrasound waves.

The ultrasound waves may increase and/or enhance an effect of the cosmetic actives/formula included in the medium 6 for the skin. Therefore, the kit of one embodiment of the invention is expected to provide perceivable cosmetic benefits of the skin.

HIFU has been primarily applied to a flat or striped localized region of the skin. On the other hand, in one embodiment of the invention, the focusing unit 4 may focus the ultrasound waves on a spot-like thin linear, or 3D-shaped region. Therefore, the kit of one embodiment of the invention is also effective for relatively small areas and is effective in improving the wrinkles of any shape and/or size in the skin.

In one embodiment of the invention, the housing 1 houses the electronic unit 2 and, the transducer 3 and the focusing unit 4 of the ultrasound waves unit 7.

In another embodiment of the invention, the housing houses the transducer and focusing unit of the ultrasound waves unit.

In one embodiment of the invention, the head 5 is close to or in contact with the skin. During use of the kit 20, the ultrasound waves travels from the head 5 through the medium 6 to the skin. The ultrasound waves may increase and/or enhance the effect of the cosmetic actives/formula for the localized region between the stratum corneum layer and the epidermal layer of the skin.

In another embodiment of the invention, the head is part of a cartridge attached to the housing. The cartridge may be removable and/or replaceable. In the embodiment, the cartridge may be replaced with a cartridge having a desired head depending on the applied location and the use of the kit. In the embodiment, when the head is dirty, only the cartridge may be removed to clean the head of the cartridge. In the embodiment, when the head is damaged, the cartridge may be replaced with a new one.

In one embodiment of the invention, the medium 6 is a cosmetic material which may be applied to the surface of the skin. The medium 6 is also a material for perceivable cosmetic benefits of the skin and for propagating ultrasound waves. Further, the skin is gently warmed by the ultrasound waves at the focal point within short time. As the skin is warmed, the effect of the cosmetic actives/formula included in the medium 6 for the skin may be increased and/or enhanced.

In one embodiment of the invention, the medium 6 is a composition including cosmetic actives/formula. The cosmetic actives/formula may include the ingredients described above.

The known HIFU for professional devices are invasive because they use ultrasound waves with high energy. Therefore, the HIFU devices can only be operated by experts or dermatologists and they are regarded as a form of medical treatment or equivalent.

On the other hand, the kit of one embodiment of the invention uses ultrasound waves with lower energy than that of the HIFU devices and thereby, the surface temperature of the applied areas of the skin is lower than that of the HIFU devices, so the kit is less invasive. Therefore, the kit of one embodiment of the invention does not require a medical license to use it and it is for a non-medical treatment. That is, the kit of one embodiment of the invention is for salons or homes.

In another embodiment of the invention, the electronic unit may further include a temperature sensor and a cooling unit. The temperature sensor detects whether the temperature of the medium applied to the skin to deliver the ultrasound waves to the skin is out of a predetermined temperature range. The cooling unit cools the medium as needed. When the control unit determines that the temperature of the medium becomes higher than the predetermined temperature range as a result of the detection by the temperature sensor, the control unit may control the driving unit so that the cooling unit is driven to cool the medium. Therefore, the kit of one embodiment of the invention has less thermal effect on the skin and is less invasive.

### Method of Using a Kit

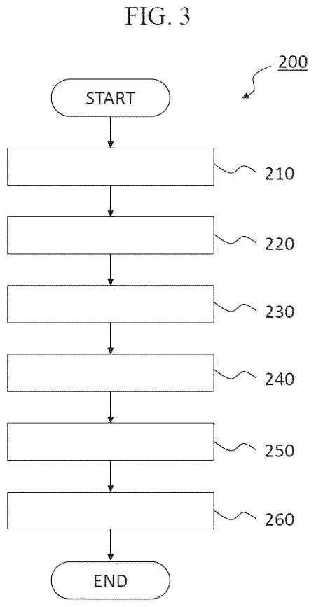

A method of using the kit of one embodiment of the invention will be described. The method of using the kit of one embodiment of the invention is a cosmetic method. That is, the method of using the kit of one embodiment of the invention is a non-therapeutic method. FIG. 3 is a block diagram showing the method of using the kit of one embodiment of the invention. In the method 200 shown in FIG. 3, the kit 20 shown in FIG. 2 is used. The method 200 includes block 210, block 220, block 230, block 240, block 250, and block 260.

In one embodiment of the invention, at block 210, the step of applying the medium 6 of FIG. 2 to the surface of the skin is performed.

In one embodiment of the invention, at the block 220, the step of controlling the driving unit based on the operation parameters using the control unit of the electronic unit 2 of FIG. 2 is performed.

In one embodiment of the invention, at the block 230, the step of driving the transducer 3 and the focusing unit 4 of the ultrasound waves unit 7 of FIG. 2 by means of the controlled driving unit is performed.

In one embodiment of the invention, at block 240, the step of generating the ultrasound waves by means of the ultrasound waves unit is performed. In one embodiment of the invention, the frequency and the energy of the ultrasound waves may be used to indirectly refer to the depth of the ultrasound waves from the surface of the skin.

In one embodiment of the invention, the ultrasound waves have a frequency of 10 KHz to 50 MHz, preferably 30 KHz to 20 MHz, more preferably 500 KHz to 10 MHz, and further more preferably 1 MHz to 10 MHz. The ultrasound waves may have the frequency of 3 MHz to 9 MHz and may have the frequency of 5 MHz to 9 MHz.

In one embodiment of the invention, the ultrasound waves have an energy of 0.001 J or more and less than 10 J, preferably 0.01 J or more and less than 5 J.

In one embodiment of the invention, the temperature of the skin at the focal zone when applying the ultrasound waves to the skin is more than 40° C. and 65° C. or lower, preferably more than 40° C. and 60° C. or lower, and more preferably more than 40° C. and 50° C. or lower.

In one embodiment of the invention, at block 250, the step of contacting the head 5 of FIG. 2 with the skin or the medium 6 on the skin is performed.

In one embodiment of the invention, at block 260, the step of propagating the ultrasound waves through the medium 6 to the skin is performed.

In this way, one embodiment of the invention can provide the method combining the medium including cosmetic actives/formula and the ultrasound waves for facial enhancement, which are of lower energy than the HIFU, the kit for implementing the method, and a method of using the kit.

In one embodiment of the invention, prior to the step of generating the ultrasound waves, the step of applying the medium 6 to the surface of the skin is performed, but is not limited to this. The step of applying the medium 6 to the surface of the skin may be performed at any time prior to the step of propagating the ultrasound waves through the medium 6 to the skin.

In another embodiment of the invention, when the ultrasound waves unit comprises the transducer only, known focusing methods are used to focus the ultrasound waves.

In yet another embodiment of the invention, the cosmetic device of the kit does not necessarily comprise a control unit and a driving unit. Therefore, the method of yet another embodiment of the invention does not include steps of controlling the driving unit and driving the transducer.

Although an example of the present invention has been presented with reference to the drawings, the present invention is not limited to the drawings. From the viewpoint of the above teachings, many modifications and variations of the present invention are possible. Therefore, within the scope of claims, the claimed invention can be implemented by methods other than those specifically described in the present specification.

### EXAMPLES

Hereinafter, the present invention will be described with reference to examples, but the present invention is not limited to these examples.

### Example 1

Hyaluronic acid serum (manufactured internally by L′Oréal) was applied to half of the face of the subject as a medium. The molecular weight of the hyaluronic acid in the hyaluronic acid serum was 50 k-1.2 MDa. The concentration of the hyaluronic acid was 1.5% relative to the total mass of the hyaluronic acid serum. The dynamic viscosity of the hyaluronic acid serum was 3.5 Pas.

After application, a kit as shown in FIG. 2 having an array of piezoelectric transducers was applied to the skin under the following conditions, and such tests were performed 5 times in succession every other day. Experts visually observed wrinkles under eyes and wrinkles at the corners of the eyes (also referred to as “crow's feet”) of the subject.

Focus depth of the focused ultrasound waves: 2.5 mm

Frequency of the focused ultrasound waves: 7.5 MHz

In Example 1, it was confirmed that the wrinkles under and at the corners of the subject's eyes were improved after one week from the last test.

### Example 2

The test was carried out in the same manner as in Example 1, except that nasolabial fine lines of the subject were further observed after each test.

### Comparative Example 2

The test was carried out in the same manner as in Example 2, except the ultrasound waves were not applied to the skin after application.

In Example 2, the effectiveness against the wrinkles under the eyes was confirmed immediately after each test. Further, the wrinkles under and at the corners of the eyes, and the nasolabial fine lines of the subject after one week from the last test were confirmed to be cumulatively improved by continuous tests. Therefore, the method of one embodiment of the invention has been shown to provide immediate and/or cumulative improvements on the wrinkles under and at the corners of the eyes, and the nasolabial fine lines.

On the other hand, the result of Comparative Example 2 showed the effectiveness against the wrinkles under and at the corners of the eyes, and the nasolabial fine lines were inferior to that of Example 2 immediately after each test.

From the above results, it was shown that the present invention is useful.

### REFERENCE SIGNS LIST

1 Housing
2 Electronic unit
3 Transducer
4 Focusing unit
5 Head
6 Medium
7 Ultrasound waves unit
10 Cosmetic device
20 Kit
100 Method for reducing wrinkles on a skin surface
110 block
120 block
200: Method of using a kit
210 block
220 block
230 block
240 block
250 block
260 block

1. A non-therapeutic cosmetic method for reducing wrinkles on a skin surface, the method comprising the steps of:
applying an ultrasound transmission medium onto the skin surface to be treated, wherein the medium includes cosmetic actives/formula and
applying ultrasound waves to the skin surface with the applied medium,
wherein the ultrasound waves produce acoustic output, wherein the ultrasound waves have a frequency of 10 KHz to 50 MHz, preferably 30 KHz to 20 MHz, and more preferably 1 MHz to 10 MHz, and
wherein the ultrasound waves are generated so that the ultrasound waves propagate in the skin to a maximum depth of 3 mm from the skin surface and generate a rise in temperature of the skin at the application zones.

2. The method according to claim 1, wherein the ultrasound waves are non-focused ultrasound waves and
wherein the temperature of the skin surface is 50° C. or lower.

3. The method according to claim 1, wherein the ultrasound waves are focused ultrasound waves and the ultrasound waves generate a local rise in temperature of the skin at the focal zone in a short time and
wherein the temperature of the skin at the focal zone when applying the ultrasound waves to the skin is more than 40° C. and 65° C. or lower, preferably more than 40° C. and 60° C. or lower, and more preferably more than 40° C. and 50° C. or lower.

4. The method according to claim 1, wherein the ultrasound waves are pulsed and focused ultrasound waves and
wherein pulse energy of the ultrasound waves is 0.001 J or more and less than 10 J, preferably 0.01 J or more and less than 5 J.

5. The method according to claim 1, wherein the ultrasound waves are pulsed ultrasound waves and
wherein pulse duration of the ultrasound waves is 150 ms or less, preferably 80 ms or less, more preferably 4 ms to 80 ms, even more preferably 4 ms to 70 ms, and yet further more preferably 4 ms to 50 ms.

6. The method according to claim 1, wherein the ultrasound waves are focused and pulsed ultrasound waves and
wherein peak power of the ultrasound waves is less than 80 W, preferably 65 W or less, more preferably 15 W or less, even more preferably 5 W or less, and yet further more preferably 0.1 W to 2 W.

7. The method according to claim 1, wherein the ultrasound waves are continuous waves and
wherein duration time of the ultrasound waves is 1 s or less.

8. The method according to claim 1, wherein the ultrasound waves is propagated to one or more layers of the skin, which are epidermis and/or dermis.

9. The method according to claim 1, wherein the cosmetic actives/formula includes at least one selected from the group consisting of hyaluronic acid, polyols, vitamins, peptides, ceramides, growth factors.

10. The method according to claim 1, wherein the cosmetic actives/formula includes hyaluronic acid and wherein a concentration of the hyaluronic acid is greater than 0.1% relative to the total mass of the medium.

11. The method according to claim 9, wherein a molecular weight of the hyaluronic acid is between 5 KDa and 20 MDa, preferably between 10 KDa and 2 MDa.

12. The method according to claim 1, wherein a dynamic viscosity of the medium is between 4 Pas and 50 Pas.

13. A kit for implementing the method according to claim 1, the kit comprising:
a device, comprising:
an ultrasound waves unit which comprises a transducer which emits ultrasound waves;
a housing which houses the ultrasound waves unit; and
a head, and
a medium for perceivable cosmetic benefits of skin and for propagating the ultrasound waves, wherein the medium includes cosmetic actives/formula.

14. A method of using a kit, the kit comprising:
a device, comprising:
an ultrasound waves unit which comprises a transducer which emits ultrasound waves;
a housing which houses the ultrasound waves unit; and
a head,
a medium for perceivable cosmetic benefits of skin and for propagating the ultrasound waves, wherein the medium includes cosmetic actives/formula;
wherein the method comprises steps of:
applying the medium to a surface of the skin;
generating the ultrasound waves by means of the ultrasound waves unit;
contacting the head with the skin or the medium on the skin; and
propagating the ultrasound waves through the medium to the skin;
wherein the ultrasound waves are focused and pulsed ultrasound waves;
wherein the ultrasound waves have a frequency of 10 KHz to 50 MHz, preferably 30 KHz to 20 MHz, and more preferably 1 MHz to 10 MHz;
wherein the ultrasound waves have energy of 0.001 J or more and less than 10 J, preferably 0.01 J or more and less than 5 J; and
wherein the temperature of the skin at the focal zone when applying the ultrasound waves to the skin is more than 40° C. and 65° C. or lower, preferably more than 40° C. and 60° C. or lower, and more preferably more than 40° C. and 50° C. or lower.
